(19) United States
(12) Patent Application Publication
(10) Pub. No.: US 2020/0089664 A1
(43) Pub. Date: Mar. 19, 2020

(54) SYSTEM AND METHOD FOR DOMAIN-SPECIFIC ANALYTICS

(71) Applicants: Channing Heard Russell, Belmont, MA (US); David Martin Fram, Watertown, MA (US); Geoffrey Edward Gordon, Gloucester, MA (US)

(72) Inventors: Channing Heard Russell, Belmont, MA (US); David Martin Fram, Watertown, MA (US); Geoffrey Edward Gordon, Gloucester, MA (US)

(73) Assignee: Commonwealth Informatics, Waltham, MA (US)

(21) Appl. No.: 16/069,050

(22) PCT Filed: Jan. 19, 2017
(86) PCT No.: PCT/US2017/014032

§ 371 Date: Jul. 10, 2018

Related U.S. Application Data
(60) Provisional application No. 62/388,191, filed on Jan. 20, 2016.

(51) Int. Cl. G06F 16/21; G16H 50/70

(52) U.S. Cl. CPC G06F 16/213; G16H 50/70

(57) ABSTRACT

Disclosed is a domain- specific data analysis system for analyzing a source database within a domain of expertise. By means of a graphical interface and a set of generic database operators, a user may construct a domain schema which is a mapping between the source database and a set of domain-specific data operators and domain-specific visualizations. The domain schema may then be used in subsequent domain-specific analyses of the source database. Even though the source database may have naming and data structure variations, domain-specific queries may be performed by users with minimal programming skills.

### CROSS-REFERENCE TO RELATED APPLICATIONS

This application claims the benefit and priority of U.S. Provisional patent application Ser. No. 62/388,191 filed Jan. 20, 2016 entitled SYSTEM AND METHOD FOR DOMAIN-SPECIFIC ANALYTICS, the entire disclosure of which is incorporated herein by reference.

### FIELD OF THE INVENTION

The present invention relates to electronic databases, and more specifically to facilitation of the analysis of databases whose contents are based on complex domain-specific entities such as databases of medical patients.

### BACKGROUND OF THE INVENTION

Electronic databases, such as relational databases, are designed to represent and store information as generic entities, such as tables (two-dimensional collections of data, with columns and rows, that are permanently stored in the database) and views (virtual collections of data which appear similar to tables but are computed by the database software as needed). The same electronic database might, for example, be used for tables and views that represent medical patients, or car parts, or airline flights, or sales orders. To make such electronic database information available for routine data entry and reporting, software applications have been developed to present the information in a way that is understandable to the application user in performing daily operations and obtaining standard reports.

Flexible data analysis tools have been developed to support the exploration of electronic databases in ways that go beyond the limited menu of services provided by typical software applications. These tools permit the design, construction, and execution of custom analyses as a series of steps. In a relational database, for example, these steps might consist of individual statements written in the SQL language, with specification of the statements via a textual or graphical user interface.

Traditional analysis tools of this kind for working with electronic databases mirror the generic nature of the electronic database itself, and are designed to work with, for example, tables of data and relationships among those tables, without concern for the nature of the real-world entities represented by the database. This allows traditional analysis tools to take advantage of the generality and efficiency of the underlying electronic database, without requiring the development of a different analysis tool for each application domain.

As a result, however, a data analysis process based on the use of traditional analysis tools can be opaque to domain specialists, whose expertise lies within the domain rather than in the construction and interpretation of generic database statements. This is especially true when the entities represented by the database are highly complex, such as medical patients. Clinicians developing, reviewing, or interpreting an analysis often prefer to work with the patients represented not only as a set of tables but also in domain-specific ways (such as through a form or dialog box specifying the desired characteristics of a set of patients or through the output of a graphical patient timeline). In this medical environment, the clinicians are the primary decision-makers who depend on the analysis of the electronic database, and inability to work with analyses in a domain-specific “physician-friendly” way impairs their ability take full advantage of the information content of the database.

There is therefore a need for a new kind of database analysis capability, namely a domain-specific data analyzer that tightly integrates generic and domain-specific operations and visualizations, so that the user can draw upon the full power of generic table-oriented database operations while simultaneously generating and viewing the results in ways that are tailored to the specific domain of use.

### SUMMARY OF THE INVENTION

Accordingly, it is a general objective of the present disclosure to create a domain-specific data analyzer and a method for performing domain-specific data analyses.

It is further an objective of the present disclosure to create a method of domain-specific data analysis allowing the user to define an arbitrarily sophisticated mapping between generic tables and domain-specific concepts in order to support the tight integration of generic and domain-specific operations and visualizations. The mapping produced by the domain-specific data analyzer is hereinafter referred to as a domain schema.

It is further an objective of the present disclosure to create a method of constructing a domain schema that allows the user to define the domain schema using only generic database capabilities.

It is further an objective of the present disclosure to optionally provide a domain-specific data analyzer operating on a server computer accessed by a client computer, without the need to install custom software on the client computer.

It should be understood that, although present disclosure makes reference to embodiments in the medical or clinical domain, the invention has broad applications in other domains and all such applications are within the scope of the present disclosure.

The present invention provides a system and method to create a new kind of analysis tool which combines the ability to work efficiently with generic tables and generic relationships among the tables while also providing a set of domain-specific operations and the ability to generate domain-specific forms of output.

The present invention addresses a significant problem in the use of electronic databases containing complex domain-specific entities by providing results in formats that are readily understood and appreciated by experts in the domain in question.

The representation of a complex entity in an electronic database typically requires the definition of a number of separate database objects (tables, in the case of a relational database), linked together by common identifiers. To continue with the clinical database example, the tables might consist of a patient demographics table, a vital signs table, a therapies table, a medical diagnoses table, a laboratory orders table, a laboratory results table, and so forth. Each of these tables characteristically contains a set of columns of data representing attributes of or measurements on multiple patients, with the association between a table row and a specific patient maintained through a column in each table that contains a unique patient identifier (such as a medical plan enrollment number or a hospital admission number).

In a generic data analysis tool, the information in this form is not in itself useful in providing domain-specific operations and outputs because the electronic database and the traditional analysis tools are not aware of the semantic meaning of the data organization and do not know about the domain-specific content of the individual tables.

An additional challenge relates to the variability of the electronic database data within a domain. In the clinical database domain, the number of, the detailed contents of, and the naming of the different tables and table columns is likely to vary considerably across different databases. An effective system and method for providing a domain-specific view of the database must be able to accommodate this variability.

The present invention addresses these challenges through the creation and application of a domain schema, which is a mapping between individual database specifics and a standardized representation of data for the chosen domain. The domain schema serves as a bridge between the generic database and a set of domain-specific data analysis operators and domain-specific data output software.

The present invention includes several main elements:
i) Computer system hardware and system software
ii) Data analysis software with generic data transformation operators
iii) Software to create and utilize one or more domain schemas
iv) Software to configure or supplement a domain schema to tailor it for a particular analysis
v) A set of domain-specific data analysis operators
vi) A set of domain-specific data output software components

It is important to note that, using the present invention, it is possible to combine freely standard data analysis steps and standard outputs with domain-specific steps and domain-specific outputs, so that the full array of standard data analysis capabilities remains available and can be intermingled with the domain-specific capabilities.

Also, it is important to note that, using this invention, there is no need for the construction of database metadata or the use of complex ontologies that would require specialized technical skills in order for the user to take advantage of the domain-specific capabilities.

### BRIEF DESCRIPTION OF THE DRAWINGS

FIG. 1 is a schematic illustration of a system for domain schema construction and domain-specific data analysis according to the present disclosure.

FIG. 2A is a schematic flowchart of a method of constructing a domain schema according to the present disclosure.

FIG. 2B is a schematic flowchart of a method of performing a domain-specific data analysis according to the present disclosure.

FIG. 3 is a schematic illustration of a system according to the present disclosure for configuring a stop button in a zero footprint environment.

FIG. 4 shows an example of a construction and analysis diagram display.

FIG. 5 shows an example of a domain-specific visualization.

FIG. 6 illustrates patient demographics definitions in an exemplary clinical schema.

FIG. 7 illustrates patient therapies definitions in an exemplary clinical schema.

FIG. 8 illustrates patient medical events definitions in an exemplary clinical schema.

FIG. 9 illustrates patient medical procedures definitions in an exemplary clinical schema.

FIG. 10 illustrates lookup table definitions in an exemplary clinical schema.

FIG. 11 illustrates an exemplary construction diagram display for constructing a domain schema according to the present disclosure.

FIG. 12 illustrates a mapping view for transforming the medical event table from native format to the columns required by the domain schema.

FIG. 13 illustrates an exemplary user interface for configuring an analysis-specific domain schema.

FIG. 14 illustrates an exemplary analysis diagram display combining generic and domain-specific operators according to the present disclosure.

FIG. 15 illustrates an exemplary specification of patient demographic details in an analysis diagram.

FIG. 16 illustrates an exemplary specification of patient drug prescription details in an analysis diagram.

FIG. 17 illustrates an exemplary specification of patient medical event details in an analysis diagram.

FIG. 18 illustrates an exemplary configuration of a temporal step in an analysis diagram.

FIG. 19 illustrates an exemplary tabular output of a domain-specific analysis.

FIG. 20 illustrates an exemplary tabular output from aggregation of results of a domain-specific analysis.

FIG. 21 illustrates an exemplary user interface showing a context menu for selecting a display of patient timeline details.

FIG. 22 illustrates an exemplary multi-patient patient timeline presentation.

FIG. 23 illustrates an exemplary single-patient patient timeline presentation.

### DETAILED DESCRIPTION OF PREFERRED EMBODIMENT

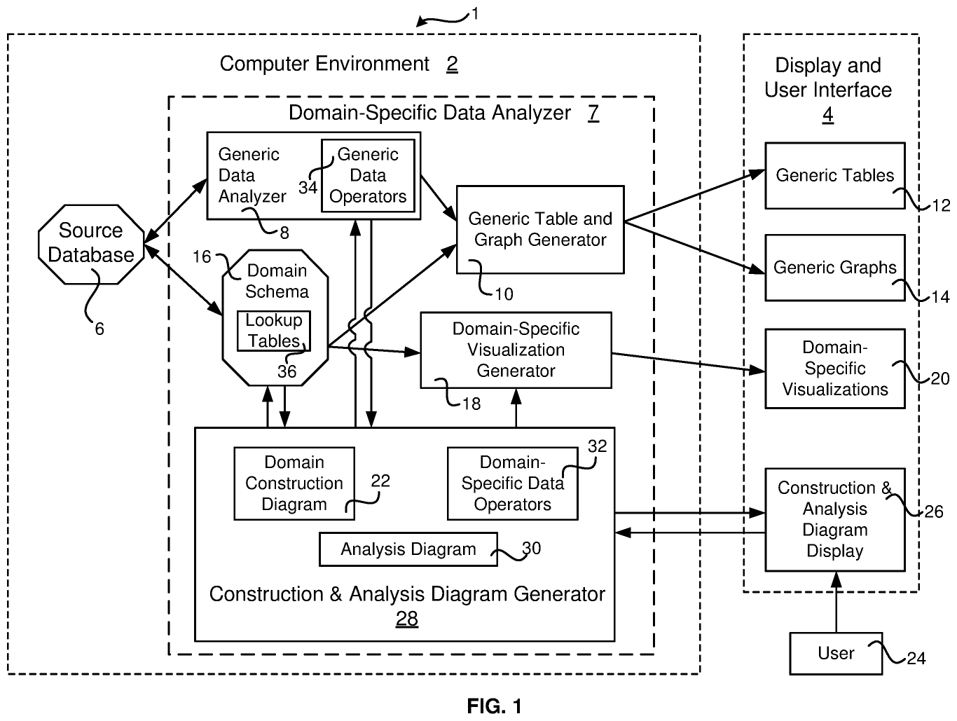

FIG. 1 is a schematic illustration of a data analysis system 1 for domain schema construction and domain-specific data analysis according to the present disclosure. Data analysis system 1 serves the dual purpose of enabling a user to construct a domain schema and enabling the user to use a previously constructed domain schema to perform a domain specific analysis of a database. Data analysis system 1 comprises a computer environment 2 and a display and user interface 4. Computer environment 2 may comprise a single computer, a client and a server within a single computer or, in a preferred embodiment, computer environment 2 may comprise a client computer and a server computer interconnected by network connections, which may be wired or wireless. Computer environment 2 further comprises a source database 6 and a domain-specific data analyzer 7. Source database 6 comprises domain data in arbitrary format. Note that source database 6 may be derived from multiple original data sources having different formats. Note also that source database 6 may include database software capable of performing operations on the data within source database 6. For example, the database software may be capable of executing instructions provided in the well-known SQL software language.

Domain-specific data analyzer 7 comprises a generic data analyzer 8, a generic table and graph generator 10, a domain schema 16, a domain-specific visualization generator 18, and a construction and analysis diagram generator 28.

The purpose of generic data analyzer 8 is to provide generic database operations, which may include instructions using the SQL software language. The generic operations are characterized by a set of generic data operators 34. By means of generic data analyzer 8 and generic table and graph generator 10, domain-specific data analyzer 7 is able to provide a broad capability for generic analysis of source database 6, including providing generic tables 12 and generic graphs 14.

Domain schema 16 is the component that provides the capability for domain-specific analysis of source database 6, and is an important part of the present invention. Domain schema 16 serves as a bridge between source database 6 and a set of domain-specific data analysis operators 32 incorporated in construction and analysis diagram generator 28, thereby providing a capability to provide domain-specific visualizations 20 on display and user interface 4. Domain schema 16 may optionally include lookup tables 36, which are tables that facilitate searching for standard domain-specific field names by cross-referencing between those names and alternative names used in database 6.

By interacting with a construction and analysis diagram display 26, a user 24 may either construct a new domain schema 16 or use an existing domain schema 16 to perform a domain-specific analysis of source database 6.

When constructing a new domain schema 16, user 24 uses generic data operators 34 to create at least one construction diagram display on display and user interface 4, and an executable domain construction diagram 22 is then generated by construction and analysis diagram generator 28. Upon executing construction diagram 22, domain schema 16 is generated, wherein domain schema 16 comprises domain-specific views of source database 6. Construction diagram 22 and domain schema 16 enable creation of a set of domain-specific data operators 32, which are able to operate on source database 6 via domain schema 16.

When using an existing domain schema 16 to perform a domain-specific analysis, user 24 creates at least one analysis diagram display using any combination of domain-specific data operators 32 and generic data operators 34. Construction and analysis diagram generator 28 then generates an executable analysis diagram 30. Upon executing analysis diagram 30, domain-specific visualization generator 18 is instructed to create domain specific visualizations 20, which are displays of data from source database 6 presented in domain-specific formats which are familiar to domain experts.

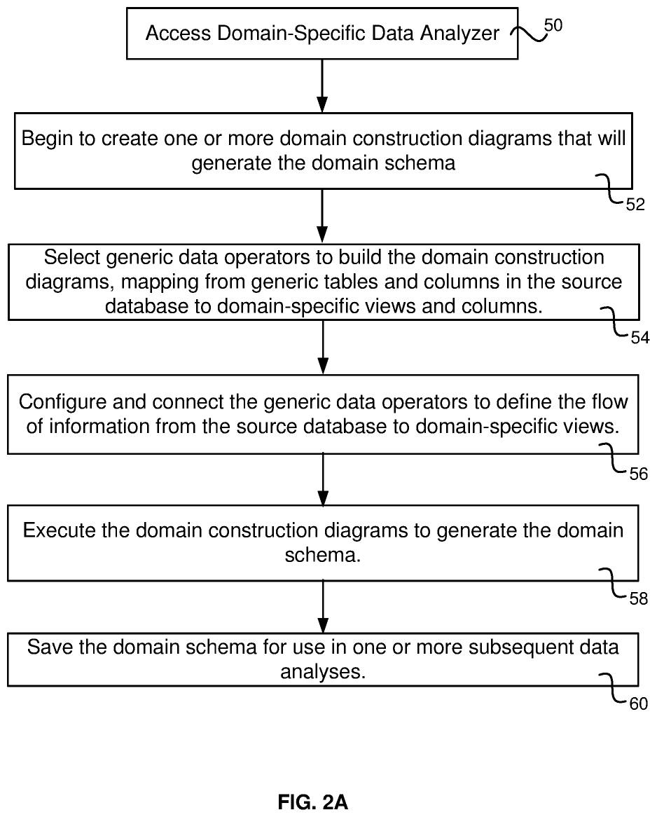

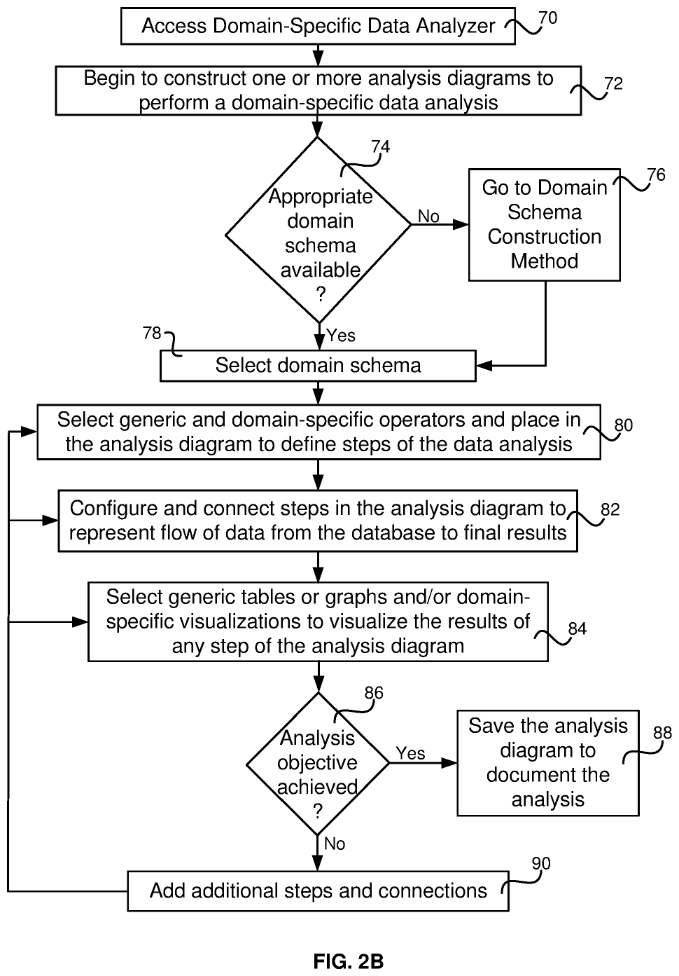

FIG. 2A is a schematic flowchart illustrating a method of constructing a domain schema according to the present disclosure. In step 50, user 24 accesses domain-specific data analyzer 7, and in step 52 begins to create one or more domain construction diagrams 22, which will generate domain schema 16. Domain construction diagrams 22 comprise mappings from generic tables and columns in source database 6 to the domain specific views and columns of domain schema 16. In step 54, domain construction diagrams 22 are built by selecting generic data operators 34, and in step 56 generic data operators 34 may be configured by double clicking on the corresponding display icon. Generic data operators 34 may also be connected to define the desired flow of information from source database 6 to domain specific views within domain schema 16. In step 58, one or more domain construction diagrams 22 are executed to generate domain schema 16, and in step 60, domain schema 16 is stored to use in one or more subsequent domain-specific data analyses.

Note that, in general, one domain construction diagram 22 may suffice for simpler domain mappings, while more than one domain construction diagram 22 may be required for more complex mappings.

FIG. 2B is a schematic flowchart illustrating a method of performing a domain-specific data analysis according to the present disclosure. In step 70, user 24 accesses domain-specific data analyzer 7, and in step 72 begins to create one or more analysis diagrams 30 to perform a domain-specific analysis of source database 6. In order to construct analysis diagram 30, a previously defined domain schema 16 is required, and availability of an appropriate domain schema is checked in step 74. If an appropriate domain schema is not available, in step 76 user 24 constructs an appropriate domain schema according to the method illustrated in FIG. 2A, and then selects that domain schema in step 78. If an appropriate domain schema is available, it is selected in step 78. In step 80, user 24 may then select an appropriate combination of generic data operators 34 and domain-specific data operators 32 and place them in analysis diagram 30 in order to define the steps of the domain-specific data analysis. In step 82 generic and domain-specific data operators may be configured by double clicking on the corresponding display icon, and operators may be connected in order to represent the desired flow of information from source database 6 to the final results of the analysis. In step 84, generic tables 12 and/or generic graphs 14 and/or domain-specific visualizations 20 may be selected to enable visualization of any step of the analysis diagram. If, in step 86, the analysis objective has been achieved then the analysis diagram is stored to document the analysis. If the objective has not been achieved, user 24 may add more steps or connections to analysis diagram 30 and may return to any of steps 80, 82 or 84 to further refine the analysis.

### The Data Analysis System

The present invention is a data analysis system which allows a user to design, develop, and execute complex multi-step analyses by assembling a set of analytical steps, interconnecting them to indicate how outputs from one step are used as inputs by other steps, and providing step configuration information where necessary. The standard steps in this data analysis environment may be implemented using a database programming language. SQL-based steps, for example, can be executed by interpreting the configuration information provided by the user and generating one or more database programming statements to perform the desired operation. Similar database techniques can be applied in non-SQL environments. Additional types of steps are provided beyond those implemented through the database programming language, which may be implemented in the data analysis software itself, to support input and output operations, including input from and output to external data files and generation of graphical presentations.

Facilities are provided for executing single steps or sets of steps in debugging or reviewing an analysis and for viewing the results of each step. In a preferred embodiment, the operations are performed by the database software within source database 6 itself, rather than by domain-specific data analyzer 7. Since in this embodiment there is no requirement to read data into the memory of domain-specific data analyzer 7, transformations and computations on large datasets may be done with high performance. In a further preferred embodiment, special techniques are provided for working with very large tables, including:
a. the ability to limit the number of rows retrieved either at the analysis level or the step level during the development of an analysis,
b. the ability to specify the creation of database indexes for result tables,
c. table display techniques designed to avoid the need to sort or count the rows in large tables,
d. an integrated batch computation facility that supports the partial or complete execution of an analysis without requiring an ongoing interaction with the user.
e. ability for the user to stop time-consuming computations, even within the execution of a single database operation, if completion is no longer desired.

In one embodiment, the data analysis environment operates as a “zero footprint” web application, in which the user works with the analysis entirely through a browser or equivalent web access software, with the application itself running either on the same computer (in the personal computer embodiment) or on a server computer (in the server embodiment). An embodiment of a zero footprint environment may be constructed using Java Server Faces (JSF) technology along with a component library (such as the PrimeFaces™ open-source library from www.primefaces.org) as building blocks, with Java Database Connectivity (JDBC) used as a mechanism for communicating with the electronic database. A preferred embodiment will maintain the current state of the analysis continually in the database itself as it is defined and executed rather than in computer main memory or in external files, which has advantages in security, reliability, and performance. It should be noted that other embodiments of a zero footprint environment may be implemented, and all such embodiments are within the scope of the present invention.

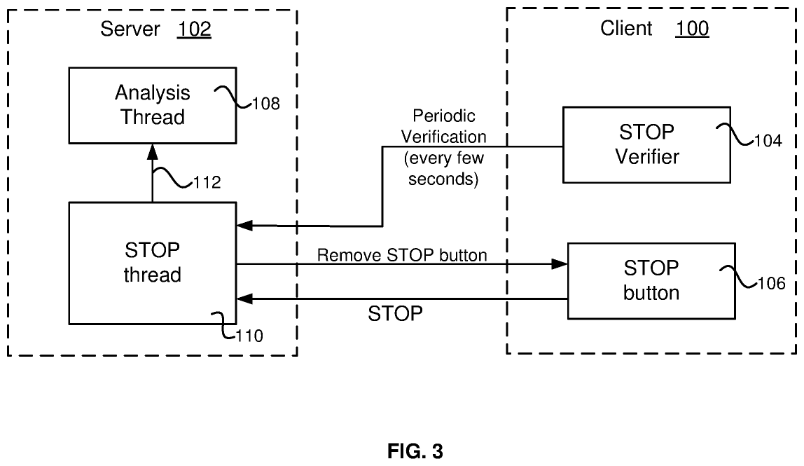

FIG. 3 is a schematic illustration of an embodiment of a system for providing a “stop button” dialog box 106 enabling the user to stop the execution of a long-running analysis step in a zero footprint environment comprising a client 100 and a server 102. Providing such a capability is challenging in a zero footprint environment because, using conventional techniques, once stop button dialog box 106 is displayed to collect user input, the software of server 102 yields control and cannot regain control until the input is provided. This would result in stop button dialog box 106 remaining on the screen after the long-running step has completed. A successful implementation of a stop button is achieved through the combination of the following elements:
a. execution of the analysis step in a background analysis thread 108,
b. a JSF dialog implementing stop button dialog box 106 that can be clicked to initiate the stopping action, and
c. a polling technique, initiated by a stop verifier 104, in which client computer 100 makes periodic remote command (“Periodic Verification”) calls to a stop thread 110 within server 102. Stop thread 110 communicates with analysis thread 108, detects successful completion of the analysis step and removes the JSF dialog containing stop button 106.

In the system for stopping analysis, the actual stop action may be implemented by the JSF dialog by sending a STOP signal from stop button 106 to stop thread 110, checking to see if analysis thread 108 stops execution, and if not then executing a cancel call for the database statement execution using the JDBC interface represented in FIG. 3 by an arrow 112.

Other embodiments for providing the user ability to stop execution may be implemented, and all such embodiments are within the scope of the present invention.

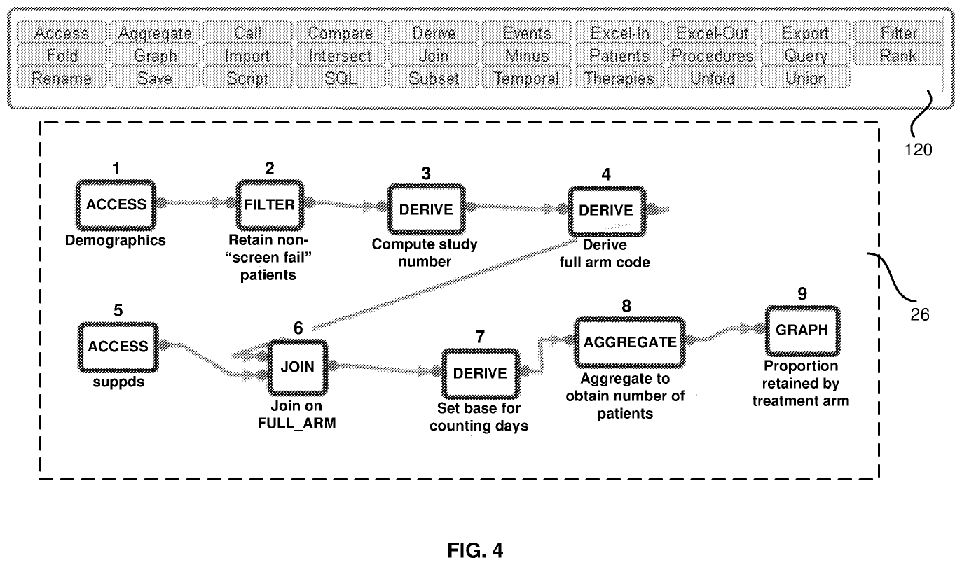

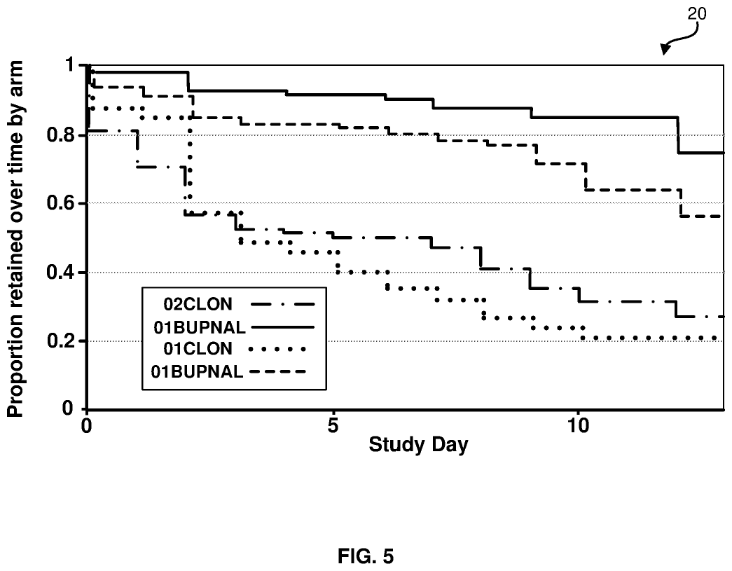

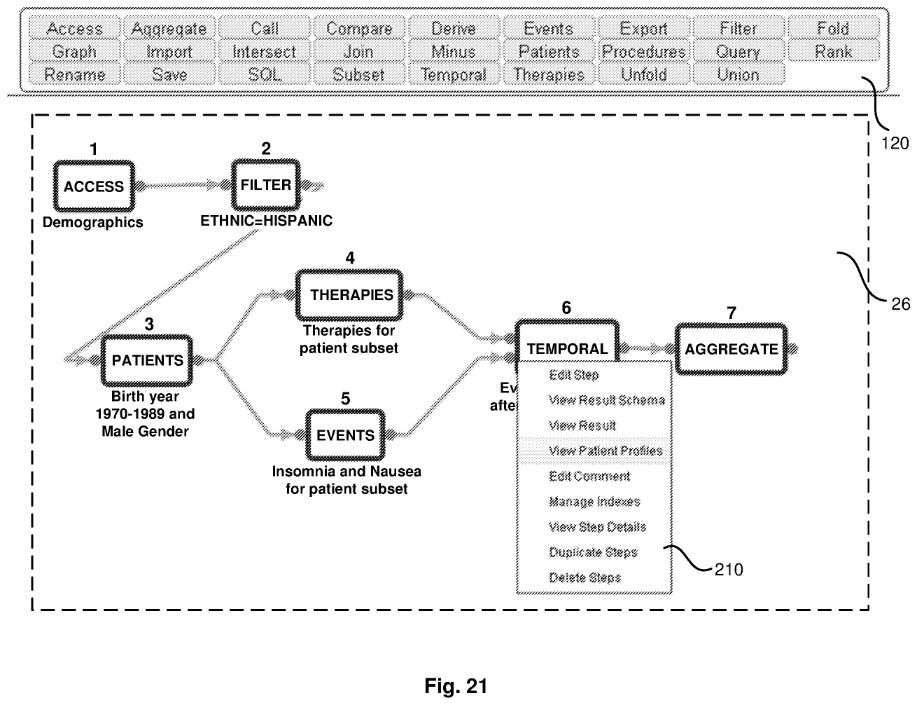

FIGS. 4 depicts an example of construction and analysis diagram display 26. Using a display such as that shown in FIG. 4, the user constructs either domain construction diagram 22 or analysis diagram 30 by linking steps defined by selecting operators in an operator selection block 120. Note that when building domain construction diagram 22, only generic data operators 34 are available in operator selection block 120, whereas when building analysis diagram 30, both generic data operators 34 and domain-specific data operators 32 are available in operator selection block 120. FIG. 5 shows an exemplary graphical result which is an example of a generic graph 14 derived from execution of the analysis diagram 30 shown in FIG. 4. The generic graph depicted in FIG. 5 is, however, making use of a domain schema and consequently domain-specific operations (“Patients”, “Procedures”, “Events” and “Temporal” in the example of FIG. 4) are available in operator selection block 120. If desired, a user may display a domain specific visualization 20 such as a patient profile (not shown) by selecting any of the steps prior to the “Aggregate” step. The process by which the user makes such a selection is described in connection with FIG. 21 below.

### Construction of the Domain Schema (Clinical Domain Example)

The primary task of domain schema 16 is to provide a mapping between domain-specific concepts and the information stored in specific electronic database tables and columns. Additionally, the domain schema may need to provide lookup tables 36 to facilitate searching fields such as drug names or diagnoses.

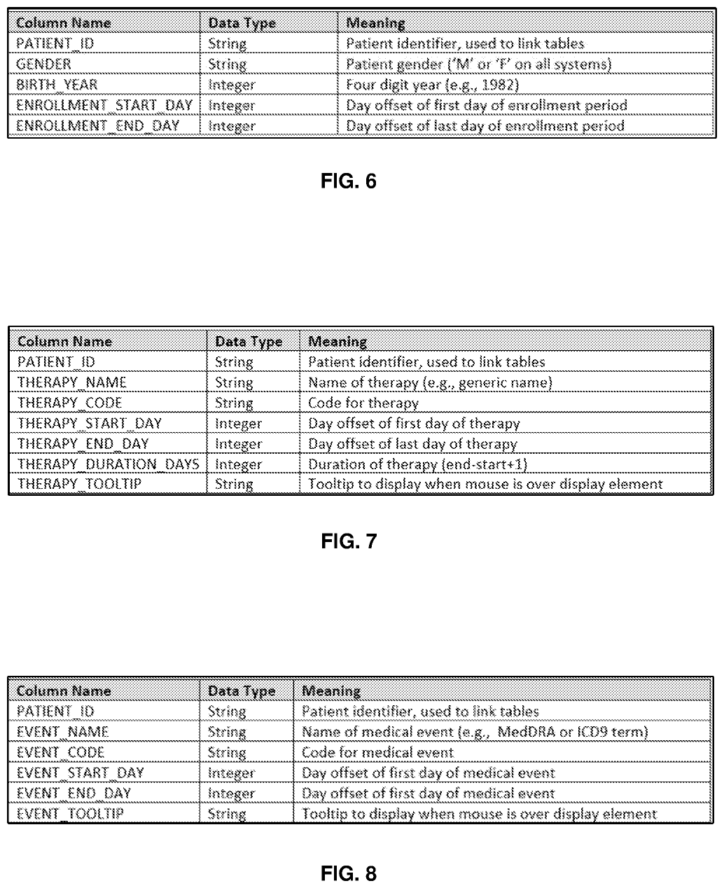

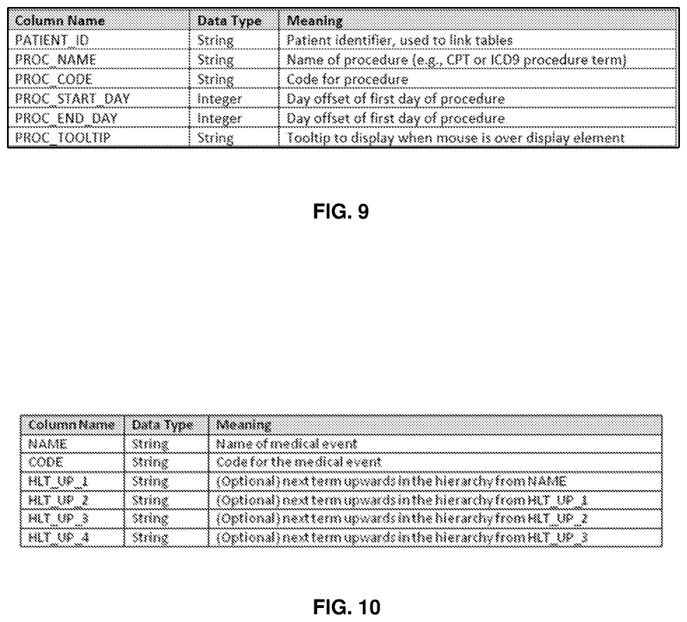

FIGS. 6, 7, 8, and 9 illustrate the domain-specific concepts to be mapped in the clinical domain example. FIG. 6 illustrates patient demographics definitions, FIG. 7 illustrates patient therapies definitions, FIG. 8 illustrates patient medical events definitions, and FIG. 9 illustrates patient medical procedures definitions. FIG. 10 is an example of a lookup table 36 depicting the concepts to be mapped for the clinical domain of medical events. Lookup tables similar to that shown in FIG. 10 perform mappings for the clinical domains of therapies and procedures.

The description below concerns methods of providing this mapping through construction of domain schema 16.

As described in connection with the method of FIG. 2A, data analysis system 1 can support the use of generic data analyzer 8 to construct tables and views that perform the mapping and provide optional lookup tables 36 associated with a particular domain schema. In the description below, the domain schema in the clinical domain is referred to as a “clinical schema”.

In the clinical example, the mapping for therapies might be based on a database view with columns using standard domain-specific names (e.g. THERAPY_NAME, THERAPY_START_DAY, THERAPY_DURATION_DAYS as shown in FIG. 7) and corresponding contents computed from the underlying database tables to return the appropriate values. This approach allows the user to develop the mapping directly using data analysis system 1, without needing intervention by a vendor of data analysis system 1 or by internal IT support staff. This mode is preferred for handling one-of-a-kind database organizations.

The clinical schema optionally includes a set of lookup tables 36 for therapies, medical events, or medical procedures. These lookup tables map user-visible names (such as the International Classification of Diseases, 9^{th} Revision (ICD9) medical event name, a descriptive text) to internal codes used in the database (such as the ICD9 code, a numeric code), and to a set of higher-level terms that categorize the names relative to a hierarchical nomenclature system. FIG. 10 shows the lookup table for the therapies described in FIG. 7. The clinical schema may also include lookup tables (not shown) for diagnoses as in FIG. 8, or for medical procedures as in FIG. 9.

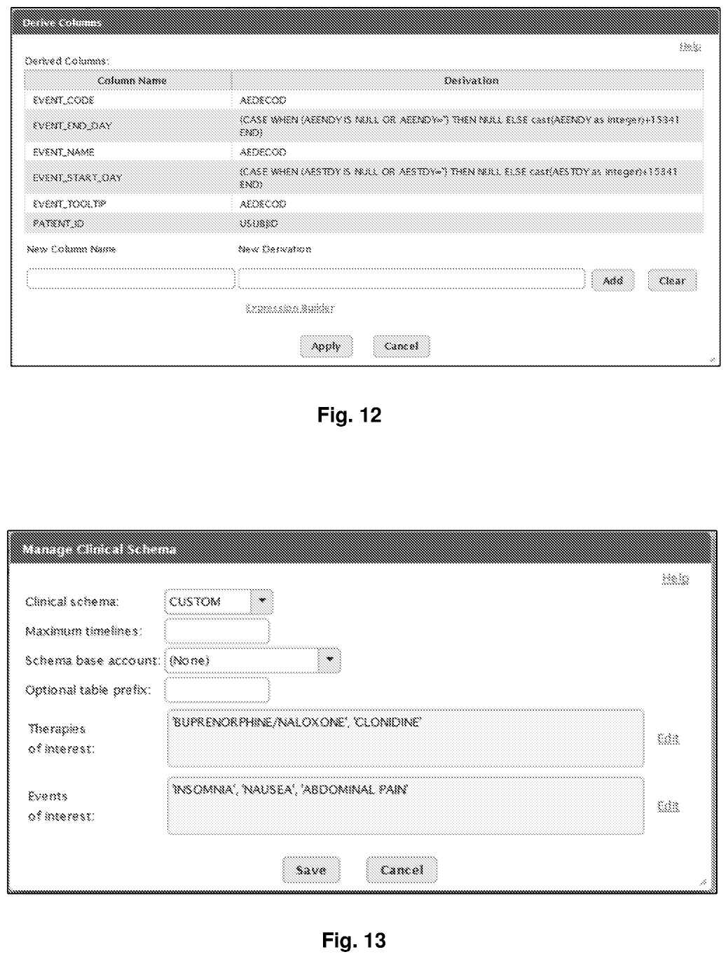

Continuing the example, an exemplary method for constructing a clinical schema is:
1. Configure a construction diagram display 26 to create a domain construction diagram 22 comprising the tables or views that define the clinical schema. The domain construction diagram 22 could be the same as the analysis diagram 30 that subsequently uses the clinical schema, or the domain construction diagram 22 could be a separate data preparation used solely for constructing the clinical schema.
2. In construction diagram display 26, utilize generic data operators 34 to read data (for example, to Access an existing table or to Import from a file) or to Derive the demographics, therapies, events, and optionally procedures tables or views (the “raw data tables”) that contain the patient data.
3. Unless the raw data tables already have the exact names of the required patient data tables/views (here, DEMOG, THERAPY, EVENT, and optionally PROC) and also already have the exact names and contents of each of the required columns, create a new view (a “mapping view”, see FIG. 12) based on each of the raw data tables. In each mapping view, create a derived column for each of the columns defined in the domain schema. While for clarity in exposition the derivations described here are simple, it is also possible utilizing this invention to construct mappings that involve many computational steps, refer to external reference data, call upon procedural computations, and the like. It may be convenient, but is not required, to have each mapping view also contain additional rows representing columns from the corresponding raw data table to facilitate use in later analyses.
4. Save each of the mapping views using the required names (here, DEMOG,

THERAPY, EVENT, PROC).
5. Optionally, Import, Access, or Derive one or more lookup tables/views corresponding to the defined structure of the lookup tables (see example in FIG. 10). The lookup tables must have the name and code columns (NAME, CODE). The lookup tables can also have one or more higher-level term columns (HLT_UP_1, HLT_UP_2, HLT_UP_3, HLT_UP_4), wherein HLT refers to a refers to a Higher Level Term in a hierarchical terminology such as the Medical Dictionary for Regulatory Activities (MedDRA).
6. To use the new clinical schema in an analysis, select CUSTOM as the clinical schema type for that analysis (see FIG. 13).

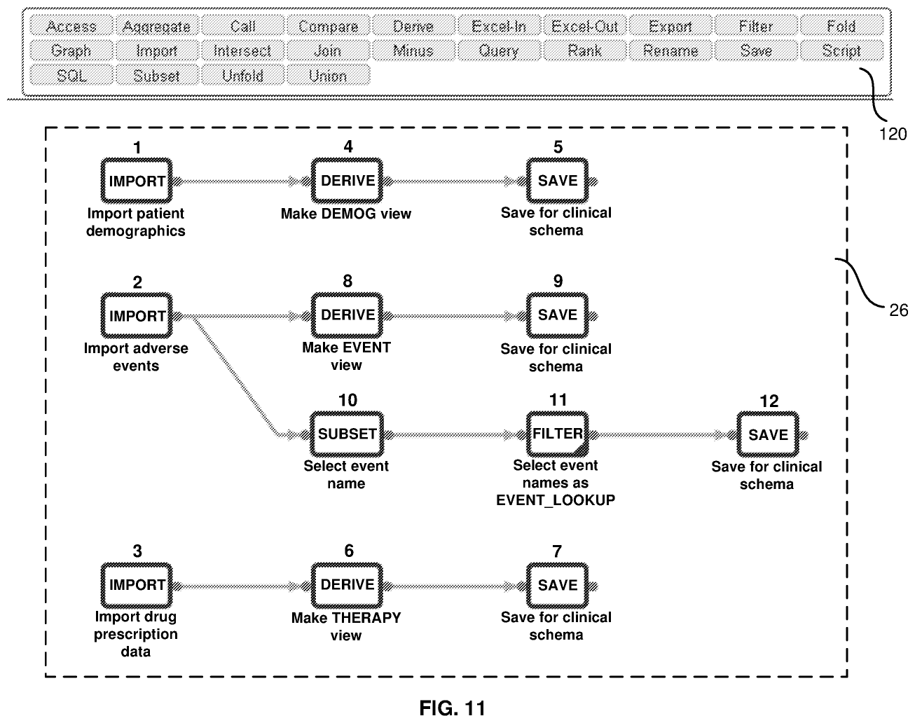

FIG. 11 illustrates the process of constructing a domain schema using the capabilities of data analysis system 1. The figure shows construction diagram display 26, for a simple clinical trial result database. Note that, in FIG. 11, operator selection block 120 comprises only generic operators 34, since a domain schema 16 has not yet been defined. Construction diagram display 26 comprises multiple interconnected operation steps, wherein each step is an application of any one of generic data operators 34, and is represented by a box, with the name of the generic operator inside the box. Below the box is a brief description of the operation. Each operation step has a step number, which is the number above the box.

In the example of FIG. 11, steps 1 to 3 import the key demographics, medical event (diagnosis), and therapy tables. These tables are “raw data tables” from source database 6 in the native format used in the clinical trial. Steps 4 and 5, steps 6 and 7, and steps 8 and 9 use generic data analyzer 8 for constructing derived columns to map the native input columns to the domain schema. FIG. 12 is a mapping view depicting the details of medical event mapping defined in Step 8 of FIG. 11. PATIENT_ID is mapped from the native USUBJID column. EVENT_NAME and EVENT_CODE defined in the domain schema are mapped from the same native column, AEDECOD, as this study did not involve assignment of codes to events. EVENT_START_DATE and EVENT_END_DATE are mapped relative to the start of the study, with special handling for blank or null values. The EVENT_TOOLTIP, used in patient profile display, is mapped from AEDECOD.

Steps 10-12 of FIG. 11 build a lookup table, EVENT_LOOKUP (shown in FIG. 10), by selecting distinct event names from the medical event data. In this particular example, the THERAPY_LOOKUP and PROC_LOOKUP lookup tables were not required.

In a more complex situation, such as the analysis of data from an electronic medical record system, multiple steps involving more complex logic would be necessary to complete construction diagram display 26. Because this invention can draw upon the full capabilities of the electronic database software included within source database 6, there is no limit to the complexity of the data transformations and computations that can be performed to construct domain schema 16 using capabilities of data analysis system 1.

Rather than relying only on its own capabilities, data analysis system 1 may optionally construct domain schema 16 by incorporating software extensions that accept terms representing domain concepts and return the appropriate data values from source database 6 for a specific database structure. In the clinical database example, these extensions might be given a term like “start of enrollment” and return the enrollment date for a specific patient.

Providing the domain schema mapping using software extensions, which become part of data analysis system 1 itself, is less flexible than constructing the mapping through generic data analyzer 8, as described above, because changes to the software extensions may require a new software release of data analysis system 1, which is undesirable. On the other hand, software extensions do provide a convenient way to build in and distribute commonly used mappings. This mode may be preferred for representing standardized database formats that change rarely. In the clinical example, such standardized database formats may include the Observational Medical Outcomes Partnership (OMOP) representation for observational databases, or the Clinical Data Interchange Standards Study Data Tabulation Model (CDISC SDTM) representation for clinical trials data. Other standardized database formats may be included, and all are within the scope of the present disclosure.

Another optional alternative way to construct domain schema 16 is to use web-based data retrieval techniques. Increasingly, web based technologies are being developed to retrieve data remotely from data repositories. In the clinical domain, one such technology is the Fast Healthcare Interoperability Resources (FHIR) standard, which defines a set of Representational State Transfer (REST) facilities that support the automated query of Electronic Health Record (EHR) systems using domain concepts. These technologies can be used to populate electronic database structures with mappings between domain-specific terms and specific values. This mode may be valuable in retrieving small amounts of data (such as, in the medical patient example, details of a single patient for use in an individual patient profile display).

### Analysis-Specific Configuration of the Domain Schema (Clinical Domain Example)

When an analysis is created in a particular domain, there must be an opportunity for the user to select a domain schema 16 appropriate to the analysis. Also, when a domain schema is used in a particular analysis, it may be useful to define additional information specific to the analysis at hand. For example, in the analysis of clinical data, it might be possible to generate more informative displays if the user were able to supplement the domain schema by indicating which particular medical events and particular therapies are of interest.

FIG. 13 illustrates how a domain schema may be selected and configured. The type of the domain schema is specified in the Clinical schema dropdown. (CUSTOM indicates that the schema was defined using the capabilities of data analysis system 1; other choices include other pre-defined schemas for standard clinical database formats such as CDISC or OMOP). Maximum timelines allows the user to specify how many multi-patient timelines will fit in the computer's browser memory. Schema base account and Optional table prefix define the physical location and naming of the domain schema tables. Therapies of interest and Events of interest allow the user to pick which therapies and events are of importance to the analysis so that these will be emphasized in subsequent domain-specific displays.

Domain-Specific Database Operators 32 (described for the Clinical Domain Example)

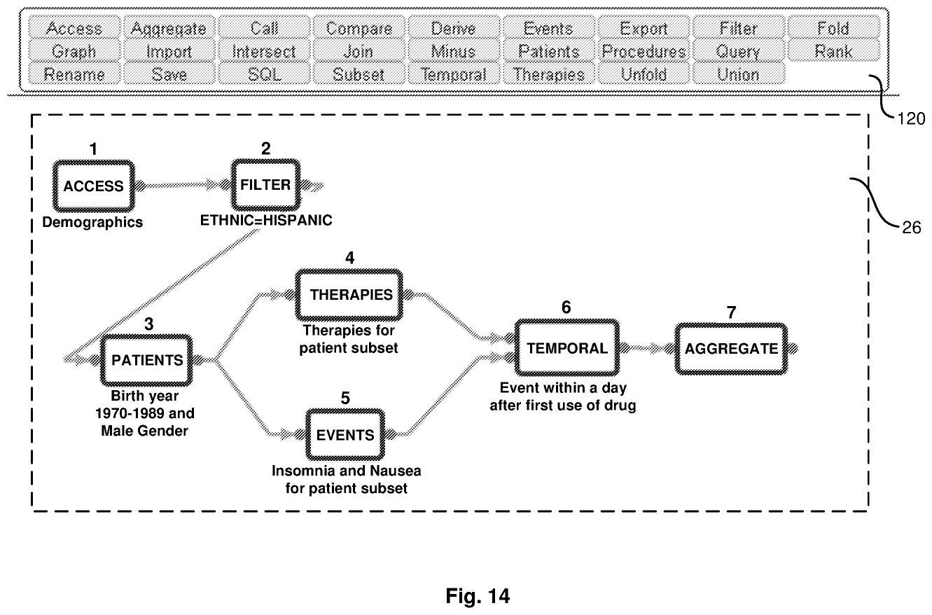

Once the user has selected and configured a domain schema 16, a set of domain-specific operators 32 becomes available in operation selection block 120 as shown in FIG. 14. In the example of FIG. 14, the domain-specific steps are Patients, Therapies, Events, Procedures, and Temporal. The objective of the following example analysis is to look for a pattern between drug dosing and adverse events by identifying all occasions in which the start of one of a set of adverse events (Insomnia or Nausea in the example analysis) occurs within one day after the start of one of a set of therapies (Buprenorphine/Naloxone and Clonidine in the example analysis).

Analysis diagram display 26 in FIG. 14 shows how these domain-specific steps can be interconnected and combined with generic data analysis steps (Access and Filter) to perform a query (a selection of a specific set of patients) and an analysis of the results of the query. This ability to intermingle generic data analysis steps with domain-specific steps is an important capability of the present invention. Here, Access and Filter are used to read in the patient data and select only Hispanic patients for inclusion.

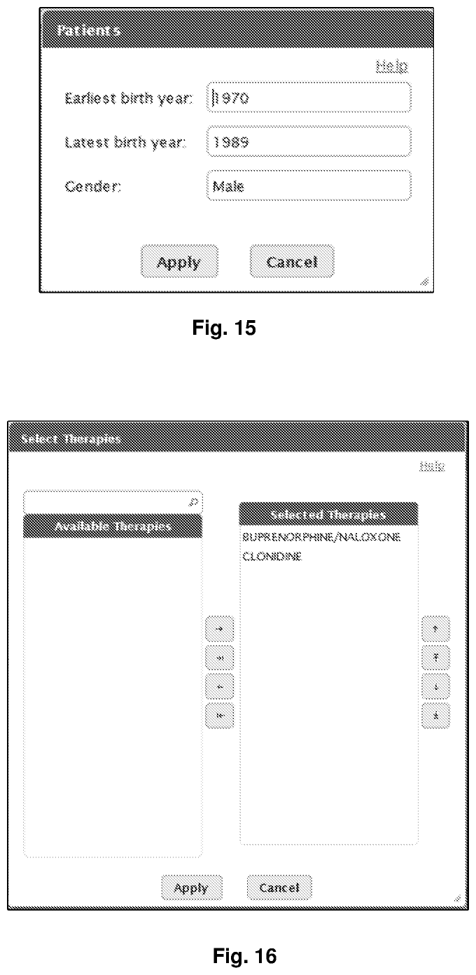

Each of the domain-specific steps has a configuration dialog that allows the user to specify the details of the computation or transformation performed by the step. FIG. 15 depicts the configuration of the Patients step of FIG. 14 by specifying the birth date range of interest and the gender of the patients. Therefore, considering the effects both of the Filter step and the Patients step, the selected subset of patients is male Hispanic patients born in the 1970's and 1980's.

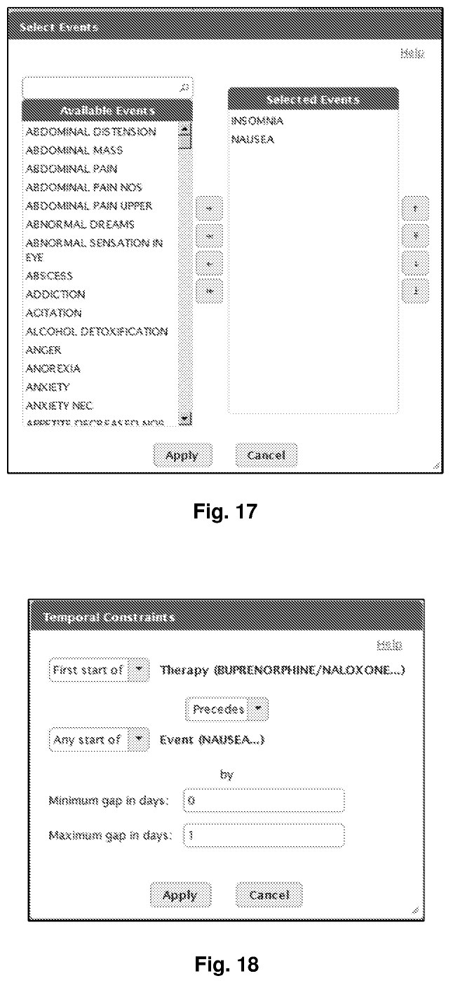

FIG. 16 depicts the configuration of the Therapies step of FIG. 14 by selecting therapies from a list of available drugs included in the study dataset. FIG. 17 depicts the configuration of the Events step by selecting adverse events from a list of available events. While in this example these selected therapies and diagnoses are the same as those defined for the analysis as therapies and diagnoses of interest (see FIG. 13), this is not a requirement.

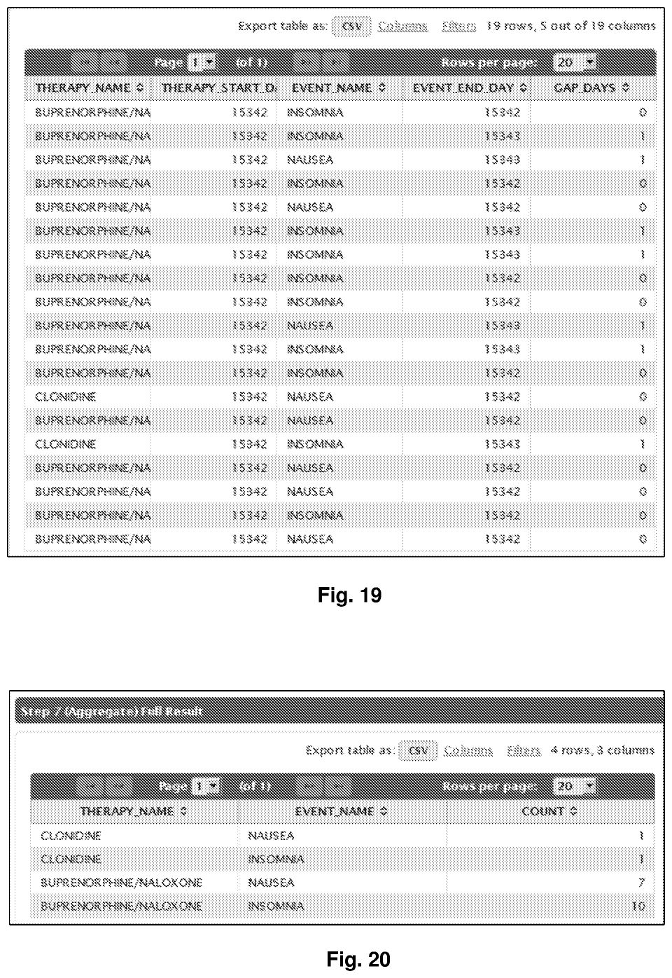

FIG. 18 shows the configuration of the Temporal step of FIG. 14, which performs a more complex temporal matching of occurrences of therapies and events within a time period. In the example shown, the user has specified a pattern that consists of the first occurrence within a patient of one of the selected therapies that precedes by no more than one day any occurrence of one of the selected events. The output of the analysis is a table listing all of the occurrences of the specified pattern, depicted in FIG. 19. This table (the result of the domain-specific Temporal query) is then used in FIG. 14 as input to a final generic data analysis step (the Aggregate step) to carry out a further analysis which counts the number of times each therapy/event combination occurs in the database. The results of the Aggregate step are depicted in FIG. 20.

Dynamic loading techniques (such as, in the Java and JSF environment, the use of class loaders or of the dynamic resource loading capability) make it possible to add new domain-specific capabilities at runtime to data analysis system 1. This makes it possible to expand the set of domain-specific capabilities supported by data analysis system 1 as needed, including support for new domains.

It is important to note that data analysis system 1 of the present invention can deal with potential naming and data structure variations in the information in source database 6. The construction of domain-specific query steps, such as those described above, is a straightforward programming task that may be performed by a domain expert with minimal programming skills.

Domain-Specific Visualizations 20 (described for the Clinical Domain Example)

In addition to the domain-specific analysis steps described above, the present invention also supports generation of domain-specific visualizations 20. As with the domain-specific analysis steps, these domain-specific visualizations are fully integrated into data analysis system 1.

FIG. 21 shows how a domain-specific output can be requested for a step, by using a context menu 210 for the step. It is important to note that context menu 210 may be selected either from a domain-specific analysis step, or from a generic analysis step as long as the tabular output of that step includes the key column that links the different domain tables (in the example of FIG. 21, the key column is PATIENT_ID). In FIG. 21, the context menu has been selected from the domain specific Temporal step, and View Patient Profiles has been selected from the context menu. Using the context menu shown, the user may also select a standard database schema view of the results of the step (View Result Schema) or a tabular display (View Result).

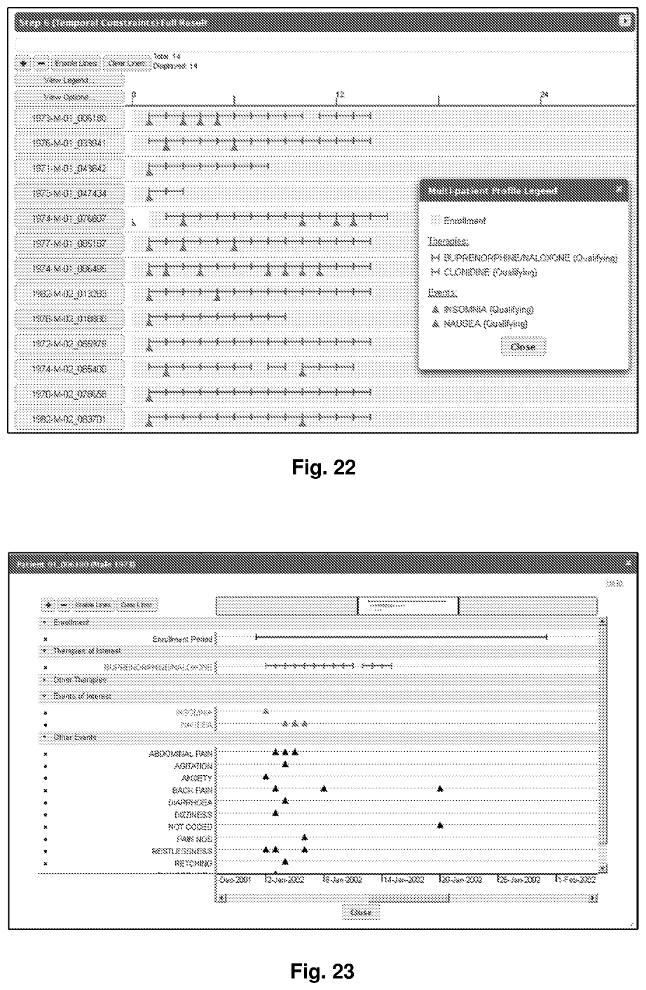

FIG. 22 depicts a domain-specific output, which is a multi-patient patient timeline display resulting from selection of “View Patient Profiles” in context menu 210 as shown in FIG. 21. In the display of FIG. 22, a row of graphical output is generated for each of the patients present in the results of the selected step. In each row of the presentation, the therapy periods for the “therapies of interest” are shown as intervals at the top of the row, and the occurrences of “events of interest” are shown as triangles at the bottom of the row. The rows present, in a format readily understood by clinicians, a concise summary of the temporal pattern of the domain-specific information that is important to the analysis.

The data sources for this display consist of the identifiers in the domain-specific linking column (here, the patient identifiers), the mappings used to create the domain schema, and the selections that were made in configuring the domain schema for this analysis. Note that it is not necessary for the user to provide any of the configuration and mapping information again for this display; the only action needed to produce the display was the selection of “View Patient Profiles” as shown in FIG. 21.

It is important to note that the ability to generate domain-specific displays quickly after any step in the analysis involving patients, without any additional setup or configuration, is a key benefit of the present invention when applied to the clinical domain. In the clinical domain, for example, this capability makes it possible for a clinician to review an analysis by inspecting patient timelines as the analysis progresses rather than searching for patient information distributed across a set of tables (for example, locating the information for the correct patient in a table of demographics, and also in a table of therapies, and also in a table of events, and so forth).

FIG. 23 depicts a detailed display of the data for one of the patient timelines, accessed by clicking on the patient identifier in one of the timelines of FIG. 22. The overall orientation of the display is similar to the multi-patient patient timelines, but it provides a much greater depth of information about the patient history (including information about therapies and events that were not listed as therapies of interest or as events of interest). This display is highly useful to a clinician in performing a detailed examination of all the therapy and event information for a patient, which might lead to the discovery of an alternative explanation for the apparent relationship between a therapy and an event. This display is generated by a single click on the patient identifier in the multi-patient patient timeline display of FIG. 22; again, because of the role of the domain schema, no setup or configuration is required to generate the display.

For simplicity of exposition, the examples of FIGS. 4 to 23 and related descriptions have been focused on a single domain, the medical patient domain. The present invention may be utilized in a variety of other domains in which the source database contains complex objects. It is particularly valuable in areas in which the key managers and decision-makers need the ability to understand the intermediate and final results of analyses in terms of domain-relevant objects rather than by piecing together information from multiple tables.

Without limitation, further examples of such domains include:
mineral exploration data (in which plots could be selected by their location and examined in geographic terms as well as in table formats in the process of performing an analysis),
mechanical engineering data (in which component parts could be selected by their key physical characteristics and displayed schematically as well as in table form in the process of performing an analysis),
data from monitoring a process manufacturing plant (in which physical components of the plant could be selected by their current in-control/out-of-control status and displayed relative to the physical or logical layout of the plant as well as in table formats in the process of performing an analysis),
data from monitoring a computer network (in which network nodes and connections could be selected based on their current utilization levels and displayed in the context of all or a portion of the network diagram as well as in table formats in the process of performing an analysis), and
data representing the maintenance history of major equipment units, such as airplanes, (in which equipment units could be selected by maintenance status and displayed showing details of the maintenance status of their components as well as in table formats in the process of performing an analysis).

Although the present invention has been described in relation to particular exemplary embodiments thereof, many other variations and modifications and other uses will become apparent to those skilled in the art. It is preferred, therefore, that the present invention not be limited by the specific disclosure

What is claimed is:

1. A database analysis system operating in a computer environment and configured to construct a domain schema and to perform a domain-specific analysis of a source database within a domain of expertise, the system comprising:
the source database;
a display and user interface comprising:
generic tables and graphs;
domain-specific visualizations;
a construction diagram display; and,
an analysis diagram display; and,
a domain-specific data analyzer comprising:
a generic data analyzer having generic data operators for operating on the source database;
the domain schema;
a construction diagram generator using the construction diagram display to generate a domain construction diagram configured to construct the domain schema and to construct domain-specific data operators for operating on the source database; and,
an analysis diagram generator using the analysis diagram display to generate an analysis diagram configured to perform a domain-specific analysis of the source database; and,
wherein a user creates the construction diagram display by connecting and configuring a multiplicity of the generic data operators; and,
wherein the user creates the analysis diagram display by connecting and configuring a multiplicity of the generic data operators and the domain-specific data operators; and,
wherein the computer environment is a zero footprint environment comprising at least one client and at least one server; and,
wherein the client includes a stop button display and a stop verifier, and the server includes an analysis thread and a stop thread, and wherein the user may click the stop button display to terminate the analysis thread, and wherein the stop verifier makes periodic remote command calls to the stop thread, causing the stop thread to verify completion or termination of the analysis thread.

2. The system of claim 1 wherein the analysis diagram generator further generates the generic tables and graphs and the domain-specific visualizations.

3. The system of claim 1 wherein the domain schema is a mapping between the source database, the domain-specific data operators and the domain-specific visualizations.

4. (canceled)

5. The system of claim 1 wherein the client and the server are implemented in a single computer.

6. The system of claim 1 wherein the client and the server are implemented in separate computers connected by a network connection.

7. (canceled)

8. The system of claim 1 wherein the zero footprint environment is constructed using a Java Server Faces (JSF) technology, with a Java Database Connectivity (JDBC) mechanism for communicating with the source database.

9. The system of claim 1 wherein the stop button display is implemented in a Java Server Faces (JSF) technology.

10. The system of claim 1 wherein the construction diagram generator makes use of an initial domain schema derived using software extensions, wherein the software extensions comprise mappings from the source database to a standardized domain-specific database format.

11. The system of claim 1 wherein the construction diagram generator makes use of an initial domain schema derived using web-based data retrieval techniques.

12. The system of claim 1 wherein a dynamic loading technique is used to enable addition of domain-specific mapping capabilities at the runtime of execution of the analysis diagram.

13. The system of claim 1 wherein the source database has naming and data structure variations, and a domain-specific query of the source database may be performed by a user with no knowledge of database programming languages.

14. The system of claim 1 wherein the domain is a clinical domain and the source database comprises human patient data.

15. The system of claim 14 wherein the domain-specific visualizations are patient timelines.

16. In a domain-specific data analysis system incorporating a generic data analyzer having generic data operators for operating on a source database within a domain of expertise, a method of constructing a domain schema comprising the steps of:
selecting a multiplicity of generic data operators to build a domain construction diagram, wherein the domain construction diagram comprises a mapping from generic tables and columns in the source database to domain-specific database views;
configuring and connecting the generic data operators to define a flow of information from the source database to the domain-specific database views;
executing the domain construction diagram to generate the domain schema, wherein the domain schema provides a mapping between domain-specific concepts and the information stored in tables and columns of the source database;
saving the domain schema for use in one or more subsequent domain-specific analyses of the source database.

17. The method of claim 16 wherein the domain is a clinical domain and the source database comprises human patient data.

18. The method of claim 16 wherein the step of configuring the generic data operators further includes a step of clicking on an icon representing a particular data operator in the domain construction diagram, thereby generating a configuration dialog, and a step of making at least one selection from the configuration dialog to specify details of the computation or transformation performed by the particular data operator.

19. The method of claim 16 wherein the step of configuring the generic data operators further includes the steps of:
clicking on an icon representing a particular data operator in the domain construction diagram, thereby generating a context menu of operations capable of being performed by the particular data operator;
selecting an operation from the context menu.

20. In a domain-specific data analysis system incorporating a generic data analyzer having generic data operators for operating on a source database within a domain of expertise and at least one domain schema having domain-specific data operators for operating on the source database, a method of performing a domain-specific analysis of the source database comprising the steps of:
selecting one of the at least one domain schema for use in the domain-specific analysis, wherein the domain schema provides a mapping between domain-specific concepts and the information stored in tables and columns of the source database;
selecting at least one generic data operator and at least one domain-specific data operator to build an analysis diagram, wherein the analysis diagram defines steps of the domain-specific analysis;
configuring and connecting the at least one generic data operator and the at least one domain-specific data operator so that the analysis diagram represents a flow of data from the source database to an analysis result.

21. The method of claim 20 wherein the analysis result comprises generic tables and graphs and domain-specific visualizations.

22. The method of claim 20 wherein the domain is a clinical domain and the source database comprises human patient data.

23. The method of claim 20 wherein the step of selecting one of the at least one domain schema further includes a step of providing domain-specific input to the domain schema.

24. The method of claim 20 wherein the step of configuring the at least one generic data operator and the at least one domain-specific data operator further includes a step of clicking on an icon representing a particular data operator in the analysis diagram, thereby generating a configuration dialog, and a step of making at least one selection from the configuration dialog to specify details of the computation or transformation performed by the particular data operator.

25. The method of claim 20 wherein the step of configuring the at least one generic data operator and the at least one domain-specific data operator further includes the steps of:
clicking on an icon representing a particular data operator in the analysis diagram, thereby generating a context menu of operations capable of being performed by the particular data operator;
selecting an operation from the context menu.

26. The method of claim 25 wherein the step of selecting an operation from the context menu is a step of selecting generic tables and graphs.

27. The method of claim 25 wherein the step of selecting an operation from the context menu is a step of selecting domain-specific visualizations.
